(19) United States
(12) Patent Application Publication
(10) Pub. No.: US 2010/0185214 A1
(43) Pub. Date: Jul. 22, 2010

(54) SANITARY DEVICE FOR REMOVAL OF BLACKHEADS AND PIMPLES

(76) Inventor: Viktoria Lönn, Osterskar (SE)

Correspondence Address: DICKSTEIN SHAPIRO LLP, 1825 EYE STREET NW, Washington, DC, 20006-5403, US

(21) Appl. No.: 12/749,067

(22) Filed: Mar. 29, 2010

Related U.S. Application Data
(63) Continuation of application No. 11/883,171, filed on Jul. 27, 2007.

(30) Foreign Application Priority Data
Mar. 3, 2005 (SE) 0500480-9

(51) Int. Cl. A61B 17/50

(52) U.S. Cl. 606/131

(57) ABSTRACT

A sanitary device for removal of blackheads and pimples. According to the invention, the sanitary device comprises two elongated sticks held in respective hands of a user, each stick formed of an elongated body having a handling portion and a treatment portion, the treatment portion being rounded and having a suitable surface roughness.

### FIELD OF THE INVENTION

The invention relates to a sanitary device for removal of blackheads and pimples.

### BACKGROUND OF THE INVENTION

Up to now blackheads and pimples are removed by the fingers of a person, and in particular the fingernails. The problem when using fingernails is that unattractive marks arise. This problem is particular annoying when blackheads and pimples are removed from visible parts of the body, and especially the face.

Another problem when using fingernail for removing blackheads and pimples is that there are a lot of bacteria under the nails, and they may infect the wound which arise when removing the blackhead and/or pimple. This may result in scar formation.

### SUMMARY OF THE INVENTION

The object of the invention is to alleviate the above-mentioned problems.

This object is achieved in that the sanitary device comprises at least one elongated body with a handling portion and at least one treatment portion, said at least one treatment portion being rounded and having a suitable surface roughness.

### BRIEF DESCRIPTION OF THE DRAWINGS

A non-limiting example of the invention will now be described with reference to the accompanying drawings, in which

FIG. 1a is a view from above of a first embodiment of a sanitary device according to the invention,

FIG. 1b is a side view of the embodiment shown in FIG. 1a,

FIG. 2 is a side view of a second embodiment of a sanitary device according to the invention,

FIG. 3 is a side view of a third embodiment of a sanitary device according to the invention, and

FIG. 4 is a side view of a forth embodiment of a sanitary device according to the invention in which the device comprises two bodies.

### DESCRIPTION OF PREFERRED EMBODIMENTS

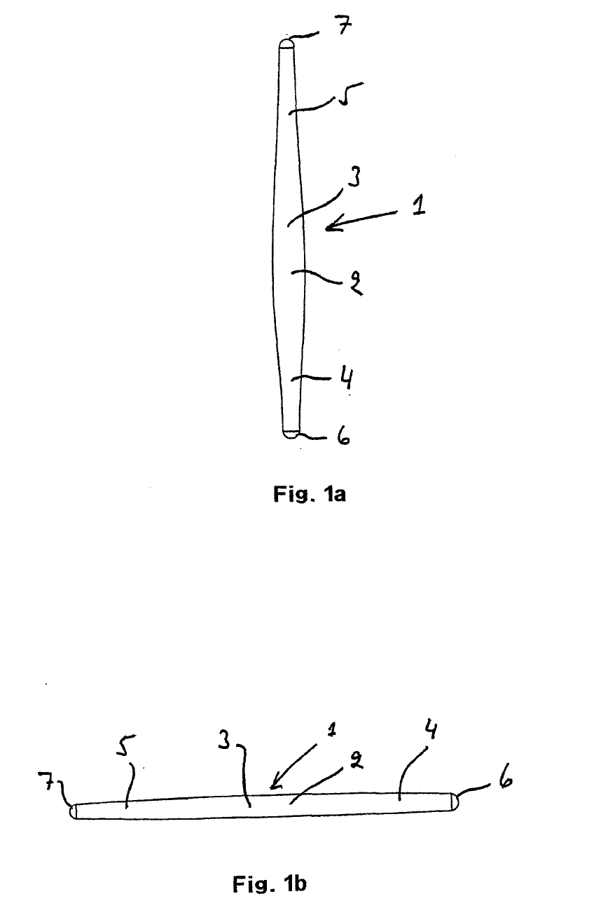

FIGS. 1a and 1b shows a sanitary device 1 according to a first embodiment comprising an elongated preferably rounded body 2 in the form of a stick having two treatment portions 4,5. The body acts as a handling portion 3. Extremities 6,7 of the treatment portions 4,5 are preferably rounded and have different surface roughness so as to increase the friction against the skin when removing blackheads and/or pimples. The diameters of the extremities 6,7 can also be different. Thus, a person using said sanitary device may choose the treatment portion 4,5 which is most appropriate for removal of said blackheads and/or pimples. Moreover, the treatment portions are preferably situated at diametrically opposed ends of the body 2.

As seen in FIG. 1a, when viewed from above, the elongated body 2 has a somewhat convex form with elongated ends, and when viewed from the side (FIG. 1b) the body 2 has rounded shape.

The handling portion 3 has preferably a particularly chosen surface structure so that a user of the sanitary device 1 will have a firm grip of said handling portion 3.

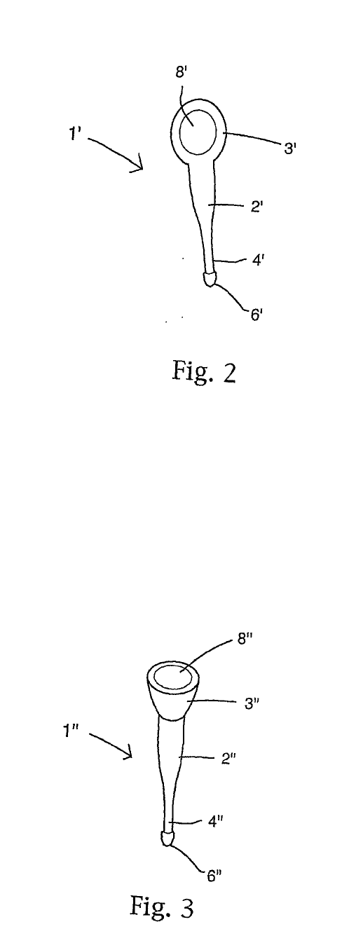

In a second embodiment shown in FIG. 2 the sanitary device 1′ comprises a elongated preferably rounded body 2′ having a handling portion 3′ and a treatment portion 4′ with a preferably rounded extremity 6′ having a suitable roughness as described above. Different sanitary devices 1′ can have rounded extremities 6′ of different diameter. The handling portion 3′ is provided with, at least on one side, a depression 8′ of a size suitable for the finger of a user which grasps the handling portion.

In a third embodiment shown in FIG. 3 the sanitary device 1″ comprises an elongated preferably rounded body 2″ having a handling portion 3″ and a treatment portion 4″ with a preferably rounded extremity 6″ having a suitable roughness as described above. As describer above different sanitary devices 1″ can have rounded extremities 6″ of different diameter. The handle portion 3″ has the form of a thimble 8″ which, in use of the sanitary device 1″, is put on the finger of a user.

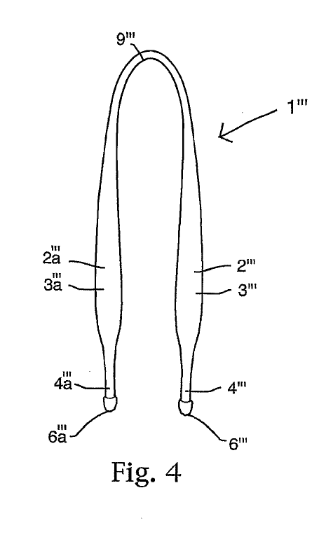

In a forth embodiment shown in FIG. 4 the sanitary device 1′″ comprises two elongated preferably rounded bodies 2″′,2″′a in the form of sticks having treatment portions 4″′,4″′a. In the same way as in the embodiment shown in FIG. 1 the bodies acts as handling portions 3′″,3′″a and the extremities 6″′,6″′a of the treatment portions 4″′,5″′a are preferably rounded. In said embodiment the bodies 2′″,2′″a are connected to each other, at the ends diametrically opposed the treatment portions 6′″,6″′a, by an elastic connecting means 9″′ of a suitable length, so that each handling portions 3′″,3′″a can be grabbed by respective hand of the user of the sanitary device 1″′ for removal of blackheads and/or pimples. In the same way as above different sanitary devices 1′″ can have rounded extremities 6″′,6″′a of different diameter.

In all of the above-mentioned embodiments the extremities 6,7;6′;6″;6″′,6′″a of the treatment portions 4,5;4′;4″;4′″,4′″a can also be provided with a surface layer (not shown)to improve the friction against the skin. The surface layer may also have antiseptic characteristics. Each treatment portions 4,5;4′;4″;4′″,4′″ can also be provided with a removable disposable tip (not shown). It is also contemplated within the invention that a set of sanitary devices 1;1′;1″;1″′ having treatment portions the extremities of which can have different surface roughness for obtaining different friction against the skin and/or different diameters of the extremity.

The sanitary device 1;1′;1″;1″′ is preferably of disposable type and made of a plastic material. However, the sanitary device 1;1′;1″;1″′ can be made of any suitable material such as wood or metal.

When using the sanitary device 1;1′;1″;1″′ according to the invention for removing blackheads and/or pimples the respective handle portions 3;3′;3″;3″′,3′″a are grabbed by each hand of the user and the extremity 6,7;6′;6″;6′″,6′″a of each treatment portion 4,5;4′;4″;4″′,4′″a is placed at each side of the blackhead and/or pimple, and by an gentle squeeze the blackhead and/or pimple is removed without leaving any marks on the skin or just miner marks which will soon disappear.

By using the sanitary device 1;1′;1″;1′″ according to the invention it is easier to remove blackheads and/or pimples situated within or behind the ears, at the wings of the nose, and the dimple in one's chin.

It should be noted that the sanitary device 1;1′;1″;1′″ according to the invention is for use in connection with normal healthy skin.

1-10. (canceled)

11. A method for removal of blackheads and pimples comprising:
providing first and second separate elongated sticks, the first and second sticks each having a treatment portion;
placing the first stick in a first hand of a user;
placing the second stick in a second hand of the user;
placing the treatment portion of the first and second sticks proximate the blackhead and/or pimple; and
using the treatment portions of the first and second sticks in the first and second hands, respectively, of the user, to squeeze the blackhead and/or pimple.

12. The method of claim 11, wherein each of the first and second sticks have a handling portion.

13. The method of claim 12, wherein the first and second hands of the user are in contact with the handling portions of the first and second sticks.

14. The method of claim 11, wherein the treatment portions the of first stick is placed on a first side of the blackhead and/or pimple and the treatment portion of the of second stick is placed on a second side of the blackhead and/or pimple, the first side of the blackhead being opposite the second side.

15. The method of claim 11, wherein the treatment portions have ball shaped ends.

16. The method of claim 15, wherein the ball shaped ends have different diameters.

17. The method of claim 15, wherein the ball shaped ends of the first and second sticks have a surface roughness different than other portions of the first and second sticks.

18. The method of claim 11, wherein the treatment portions are provided with removable disposable tips.

19. The method of claim 12, wherein the handling portions are provided with depressions of a size suitable for the finger of a user.

20. The method of claim 12, wherein the handling portions have the form of a thimble.

21. The method of claim 15, wherein the ball shaped end of the first stick has a surface roughness different from that of the ball shaped end of the second stick.

22. The method of claim 11, wherein ends of the treatment portions are provided with a surface layer having antiseptic characteristics.

23. The method of claim 11, wherein the treatment portions are on a first side of the first and second sticks and additional treatment portions are on a second side of the first and second sticks.

24. A method for removal of blackheads and pimples comprising:
providing first and second separate elongated sticks, the first and second sticks each having a handling portion and a treatment portion, the treatment portions having ball shaped ends;
placing the handling portion of the first stick in a first hand of a user;
placing the handling portion of the second stick in a second hand of the user;
placing the ball shaped ends of the first and second sticks proximate the blackhead and/or pimple; and
using the ball shaped ends of the first and second sticks in the first and second hands, respectively, of the user to squeeze the blackhead and/or pimple.

25. The method of claim 24, wherein the ball shaped ends of the first and second sticks have a surface roughness different than the handling portions of the first and second sticks.

26. The method of claim 24, wherein the ball shaped ends have different diameters.

27. The method of claim 24, wherein the treatment portions are provided with removable disposable tips.
